(19) United States
(12) Patent Application Publication
(10) Pub. No.: US 2025/0041232 A1
(43) Pub. Date: Feb. 6, 2025

(54) MICROSPHERES COMPRISING HIGH-DOSE VARENICLINE, METHOD FOR PREPARING SAME, AND PHARMACEUTICAL COMPOSITION COMPRISING SAME

(71) Applicants: AULBIO CO., LTD., Seongnam-si,Gyeonggi-do (KR); AULBIO CO., LTD., Suwon-si,Gyeonggi-do (KR)

(72) Inventors: Cherng Ju KIM, Suwon-si, Gyeonggi-do (KR); Taekun AN, Yongin-si, Gyeonggi-do (KR); Aram KIM, Seoul (KR); Hye Jin SEO, Seoul (KR); Kwi Seok YEOM, Hanam-si, Gyeonggi-do (KR); Yuri PARK, Suwon-si, Gyeonggi-do (KR)

(73) Assignees: AULBIO CO., LTD., Seongnam-si,Gyeonggi-do (KR); AULBIO CO., LTD., Suwon-si,Gyeonggi-do (KR)

(21) Appl. No.: 18/709,351

(22) PCT Filed: Dec. 9, 2021
(86) PCT No.: PCT/KR2021/018637

§ 371 Date: May 10, 2024

(30) Foreign Application Priority Data
Nov. 11, 2021 (KR) 10-2021-0155112

(51) Int. Cl. A61K 9/50; A61K 31/55

(52) U.S. Cl. CPC A61K 9/5031; A61K 9/5089; A61K 31/55

(57) ABSTRACT

Microspheres containing varenicline or a pharmaceutically acceptable salt thereof, and a biocompatible polymer are disclosed. And, a method for preparing the microspheres and a pharmaceutical composition containing the microspheres are disclosed. The microspheres containing varenicline exhibit a stable drug release rate over a long period of time and can maintain an effective concentration of varenicline in the blood for a certain period of time, thereby extending a drug administration cycle, and can increase a patient's medication compliance and reduce side effects caused by rapid initial burst of the drug.

### TECHNICAL FIELD

The present application claims the benefit of priority based on Korean Patent Application No. 10-2021-0155112 filed on Nov. 11, 2021, and all contents disclosed in the document of the Korean Patent Application are incorporated as part of this specification.

The present invention relates to microspheres comprising high-dose varenicline, a method for preparing the same, and a pharmaceutical composition comprising the same.

### BACKGROUND ART

Varenicline is a compound whose compound name is 7, 8, 9, 10-tetrahydro-6, 10-methano-6H-pyrazino[2, 3-h][3]benzazepine and has a structure represented by Formula I below.

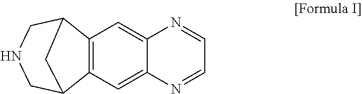 [SMILES: c1cnc2cc3c(cc2n1)C1CNCC3C1]

Varenicline, represented by Formula I, is a drug useful for improving symptoms caused by cholinergic receptor activation disorders by binding to the specific receptor site of neuronicotinic acetylcholine, and the drug is useful in the treatment of inflammatory bowel disease, irritable bowel syndrome, spastic dystonia, chronic pain, acute pain, non-tropical sprue, appendicitis, vasoconstriction, anxiety disorder, panic disorder, depression, bipolar disorder, autism, sleep disorders, jet lag syndrome, amyotrophic lateral sclerosis (ALS), cognitive dysfunction, drug/toxicity-induced cognitive impairment, disease-induced cognitive impairment, hypertension, bulimia nervosa, anorexia, obesity, cardiac arrhythmias, gastric acid hypersecretion, ulcers, pheochromocytoma, progressive supranuclear palsy, chemical dependence and addiction (For example, dependence or addiction to nicotine or tobacco products, alcohol, benzodiazepines, barbiturates, opioids, or cocaine), headaches, migraines, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including absence seizures, attention deficit hyperactivity disorder (ADHD), or Tourette syndrome, and in the treatment of nicotine dependence, addiction, and withdrawal, especially for use in smoking cessation therapy.

Currently, a smoking cessation treatment based on varenicline tartrate salt is sold worldwide under the brand name Champix® or Chantix®. Champix® is a partial agonist for α4β2 neuronal nicotine receptors and is an adjunctive therapy for smoking cessation that relieves smoking cravings and withdrawal symptoms by binding to acetylcholine receptors in the brain instead of nicotine.

The Champix® is known to have common side effects such as nausea, insomnia, constipation, abdominal distention, and vomiting. In particular, nausea is temporary, but for some patients, it is difficult to continue taking the medicine, and problems such as persistent nausea occur. Clinical trials have also shown that these symptoms increase in a dose-dependent manner. To solve this problem, the drug is taken through dose titration. First, take 0.5 mg of varenicline once a day for 3 days, and then take 0.5 mg twice a day from the 4th to the 7th day. Afterwards, the effective blood concentration was maintained and withdrawal symptoms were alleviated by taking 1.0 mg of varenicline twice a day until the 12th week.

However, this complex administration method has the disadvantage of significantly reducing medication compliance. Therefore, there is a need for the development of a sustained-release preparation that contains varenicline as the main ingredient and can improve convenience of administration.

### PRIOR ART DOCUMENTS

### Patent Document

Korean Patent Registration No. 10-0551184

### DISCLOSURE

### Technical Problem

As a result of repeated research to solve the above problems of the prior art, the present inventors developed microspheres that encapsulate varenicline at a high concentration and release it continuously for a long period of time.

Therefore, an object of the present invention is to provide microspheres in which varenicline is encapsulated in high concentration and released continuously for a long period of time, a method for preparing the same, and a pharmaceutical composition comprising the same.

### Technical Solution

In order to solve the above problems, the present invention provides microspheres comprising varenicline or a pharmaceutically acceptable salt thereof, and a biocompatible polymer.

In addition, the present invention provides a method for preparing microspheres comprising the following steps of:
(a) preparing a dispersed phase by dispersing varenicline pamoate salt and a biocompatible polymer in one or more solvents;
(b) adding the prepared dispersed phase to the continuous phase and stirring to form microspheres; and
(c) removing the solvent.

In addition, the present invention
provides a pharmaceutical composition for the prevention or treatment of diseases caused by cholinergic receptor activity disorders, comprising the microspheres of the present invention.

### Advantageous Effects

The microspheres of the present invention provide the effect of significantly improving the encapsulation rate of varenicline by encapsulating varenicline or a pharmaceutically acceptable salt thereof in a biocompatible polymer.

In addition, the microspheres of the present invention provide the effect of continuously releasing varenicline for a long period of time without initial burst.

In addition, the method for preparing microspheres containing varenicline pamoate salt of the present invention provides the effect of efficiently preparing the microspheres.

In addition, the pharmaceutical composition of the present invention provides excellent effects in preventing and treating diseases caused by cholinergic receptor activity disorders by including the microspheres.

### DESCRIPTION OF DRAWINGS

FIG. 1 is a photograph showing the results of observing the shape of the microspheres prepared in Example 2, Comparative Example 1, and Comparative Example 2 of the present invention using a scanning electron microscope (SEM), and

FIG. 2 is a graph showing the results of an experiment evaluating the in vitro release of varenicline from the microsphere encapsulated with varenicline pamoate salt (Example 2).

### BEST MODE

Hereinafter, the present invention will be described in more detail.

All technical terms used in the present invention, unless otherwise defined, are used with the same meaning as commonly understood by a person skilled in the art in the field related to the present invention. In addition, things similar or equivalent to those described as preferred methods or samples in the present invention are also included in the scope of the present invention. The contents of all publications incorporated by reference herein are hereby incorporated by reference in their entirety.

The present invention relates to microspheres comprising varenicline or a pharmaceutically acceptable salt thereof, and a biocompatible polymer.

The varenicline is a compound whose compound name is 7, 8, 9, 10-tetrahydro-6, 10-methano-6H-pyrazino[2, 3-h][3]benzazepine, and has a structure represented by the following Formula I.

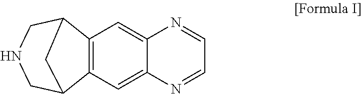 [SMILES: c1cnc2cc3c(cc2n1)C1CNCC3C1]

The varenicline is a drug useful for improving symptoms caused by cholinergic receptor activation disorders by binding to the specific receptor site of neuronicotinic acetylcholine. The drug is useful in the treatment of inflammatory bowel disease, irritable bowel syndrome, spastic dystonia, chronic pain, acute pain, non-tropical sprue, appendicitis, vasoconstriction, anxiety disorder, panic disorder, depression, bipolar disorder, autism, sleep disorders, jet lag syndrome, amyotrophic lateral sclerosis (ALS), cognitive dysfunction, drug/toxicity-induced cognitive impairment, disease-induced cognitive impairment, hypertension, bulimia nervosa, anorexia, obesity, cardiac arrhythmias, gastric acid hypersecretion, ulcers, pheochromocytoma, progressive supranuclear palsy, chemical dependence and addiction (For example, dependence or addiction to nicotine or tobacco products, alcohol, benzodiazepines, barbiturates, opioids, or cocaine), headaches, migraines, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including absence seizures, attention deficit hyperactivity disorder (ADHD), or Tourette syndrome. It is particularly useful in the treatment of nicotine dependence, addiction, and withdrawal, including use in smoking cessation therapy.

The varenicline or a pharmaceutically acceptable salt thereof may be contained in an amount of 0.1 to 40% by weight, preferably 10 to 40% by weight, more preferably 15 to 40% by weight, and even more preferably 18 to 40% by weight, relative to the total weight of the microspheres, based on the varenicline free base.

If the content of varenicline free base contained in the microspheres exceeds 40% by weight, the initial release amount of varenicline in the body environment may be excessively high, causing a problem in which the blood concentration of the drug rises rapidly. If the content of varenicline contained in microspheres is less than 0.1% by weight, the biocompatible polymer ratio becomes relatively high, making it difficult to release varenicline.

In the present invention, the varenicline free base content may have an upper limit of 30% by weight or 25% by weight depending on the dosage form.

In one embodiment of the present invention, the varenicline or a pharmaceutically acceptable salt thereof may be varenicline pamoate salt.

The varenicline pamoate salt can be prepared by conventional techniques such as adding acid to free varenicline to convert it into an acid addition salt, or converting one acid addition salt into another salt. For example, the varenicline pamoate salt can be prepared by dissolving varenicline in an aqueous acetic acid solution and adding pamoate salt to crystallize it.

The varenicline pamoate salt exhibits the same pharmacological activity as previously reported for free varenicline and other acid addition salts.

In one embodiment of the present invention, the varenicline pamoate salt contained in the microspheres may be included in an amount of 0.1 to 60% by weight, preferably 5 to 60% by weight, more preferably 10 to 60% by weight, even more preferably 15 to 60% by weight, and most preferably 17 to 60% by weight, based on the total weight of the microspheres.

If the content of varenicline pamoate salt contained in the microspheres exceeds 60% by weight, the initial release amount of varenicline in the body environment may be too high, causing a problem in which the blood concentration of the drug rises rapidly. If the content of varenicline pamoate salt contained in microspheres is less than 0.1% by weight, the biocompatible polymer ratio increases relatively, making it difficult to release varenicline.

In the present invention, the content of varenicline pamoate salt may have an upper limit of 50% by weight, 40% by weight, or 30% by weight depending on the dosage form.

In the present invention, microspheres means the varenicline pamoate salt encapsulated in microspheres manufactured using biocompatible polymer, and are simply referred to as varenicline-containing microspheres, varenicline microspheres, or microspheres. If varenicline pamoate salt is encapsulated in microspheres manufactured using a biocompatible polymer, they are all included within the scope of the present invention, regardless of the type of biocompatible polymer used.

In the present invention, the “biocompatible polymer” refers to a polymer that is safe in vivo and does not cause high cytotoxicity and inflammatory reactions when administered in vivo, and is also referred to simply as polymer in this specification.

In the present invention, biocompatible polymers can be selected based on intrinsic viscosity. A suitable intrinsic viscosity is 0.1 to 1.9 dL/g, preferably 0.1 to 1.4 dL/g, and more preferably 0.1 to 1.2 dL/g. For biocompatible polymers with an intrinsic viscosity of less than 0.1 dL/g, the degradation of the polymer is too fast, so it may be difficult to continuously release varenicline for the desired time. Biocompatible polymers with an intrinsic viscosity exceeding 0.9 dL/g may not exhibit medicinal efficacy due to slow polymer degradation and low release of varenicline.

In the present invention, the biocompatible polymer may be contained in an amount of 40 to 99.9% by weight, preferably 40 to 95% by weight, more preferably 40 to 90% by weight, even more preferably 40 to 85% by weight, and most preferably 40 to 83% by weight, based on the total weight of the microspheres. If the biocompatible polymer is contained in less than 40% by weight, the distribution of varenicline pamoate salt is relatively increased, which may cause problems with initial excessive release or inability to maintain drug efficacy for the desired period. If it is contained in excess of 99.9% by weight, the amount to be administered to the patient becomes too large, making administration difficult or administration itself impossible.

The biocompatible polymer compound may be one or more selected from the group consisting of polyglycolic acid, polylacetic acid, polyglycolide, polylactide, polylacetic-co-glycolic acid, polylactide-co-glycolide (PLGA), polyphosphazine, polyiminocarbonate, polyphosphoester, polyanhydride, polyorthoester, copolymers of lactic acid and caprolactone, polycaprolactone, polyhydroxyvalate, polyhydroxybutyrate, polyamino acids, and copolymers of lactic acid and amino acids, and most preferably polylactide-co-glycolide (PLGA).

In the present invention, the microspheres may contain impurities (ingredients excluding varenicline or its pharmaceutically acceptable salt, and biocompatible polymer) contained during the manufacturing process in an amount of 5% by weight or less based on the total weight of the microspheres. When considering such impurities, the content of the biocompatible polymer may be reduced by the content of the impurities.

According to one embodiment of the present invention, varenicline pamoate salt was confirmed to provide a high encapsulation efficiency of more than 90%.

As described above, when the encapsulation efficiency is high, it is preferable because it can be easily administered to patients by reducing the dosage, thereby improving the convenience of administration. In addition, since a larger amount of varenicline can be included in a small amount of microspheres, mass production is possible, which can be very advantageous in improving productivity.

In one embodiment of the present invention, the microspheres can provide encapsulated varenicline in a controlled or sustained release form. The above controlled or sustained release form can be understood as having the same meaning as “sustained release”, “controlled release” or “delayed release”.

In one embodiment of the present invention, the microspheres may have the characteristic that the release of encapsulated varenicline in an in vitro environment lasts for more than 30 days, and more preferably for more than 35 days.

In addition, the microspheres may have the characteristic that the varenicline contained in the microspheres is released in an in vitro environment at 60% or less, more preferably at 50% or less, within 15 days, and the remainder is continuously released after 15 days.

In addition, the microspheres may have the characteristic that the varenicline contained in the microspheres is released in an in vitro environment at 70% or less, more preferably at 60% or less, within 20 days, and the remainder is continuously released after 20 days.

In one embodiment of the present invention, the microspheres containing varenicline pamoate salt are prepared by solvent evaporation or extraction using an emulsion, more preferably, an O/W type solvent evaporation method that produces an oil-in-water (O/W) type emulsion containing a biocompatible polymer, varenicline pamoate salt, and dispersion solvent and agglomerates it into fine particles.

In the present invention, when the microspheres containing varenicline pamoate salt are manufactured according to the solvent evaporation method or solvent extraction method using an O/W type emulsion among various microsphere manufacturing methods known in the art (e.g., O/W type, O/O type or W/O/W type solvent evaporation or solvent extraction method, microspheres production method by spray drying, microspheres production method by phase separation, etc.), the encapsulation efficiency of varenicline pamoate salt within the microspheres can be significantly improved.

In order to manufacture microspheres by preparing the O/W type emulsion and agglomerating it into polymer fine particles, first, an O/W type emulsion containing a biocompatible polymer, varenicline pamoate salt, and dispersion solvent is prepared.

For the production of the O/W type emulsion, conventional methods known in the art can be used. More specifically, to prepare an O/W type emulsion, a dispersed phase containing a biocompatible polymer and varenicline pamoate salt can be added to the dispersion solvent. These polymer particles containing varenicline pamoate salt are manufactured by agglomerating the emulsion into fine particles by solvent evaporation and/or solvent extraction, or by agglomeration by ammonolysis or hydrolysis process. In the case of the ammonolysis process, by the addition of ammonia, and in the case of the hydrolysis process, by the addition of an acid or base, a water-insoluble organic solvent that is converted to a water-soluble solvent through an ammonolysis or hydrolysis reaction is additionally included in the preparation of the emulsion.

In the case of the solvent evaporation method (for example, but not limited thereto, the method described in U.S. Pat. Nos. 6,471,996, 5,985,309, and 5,271,945, etc.), after dispersing or dissolving the drug in an organic solvent in which a polymer compound is dissolved, an O/W type emulsion is prepared by emulsifying it in a dispersion medium such as water, and the organic solvent in the emulsion is diffused into the dispersion medium and evaporated through the air/water interface. By doing so, polymer microspheres containing varenicline pamoate salt can be formed.

The solvent extraction method includes a common solvent extraction method used in the production of polymer microspheres containing varenicline pamoate salt, such as effectively extracting the organic solvent in the emulsion droplets using a large amount of solubilizing solvent.

As a method of simultaneously applying the solvent evaporation method and the solvent extraction method, for example, the methods described in U.S. Pat. Nos. 4,389,840, 4,530,840, 6,544,559, 6,368,632, and 6,572,894, etc. can be applied.

Agglomeration by the ammonolysis process refers to a method of agglomerating fine particles by adding ammonia to an O/W type emulsion containing a water-insoluble organic solvent to induce ammonolysis to convert the water-insoluble organic solvent into a water-soluble solvent, such as the method described in Republic of Korea Patent No. 918092.

Agglomeration by the hydrolysis process refers to a method of agglomerating fine particles by adding base solutions such as NaOH, LiOH, KOH or acid solutions such as HCl, H₂SO₄ to an O/W type emulsion containing a water-insoluble organic solvent to induce hydrolysis, a type of hydrolysis reaction of ester, to convert the water-insoluble organic solvent into a water-soluble solvent, such as the method described in Korean Patent Application Nos. 2009-109809 and 2010-70407.

In addition, the present invention relates to a method for preparing microspheres comprising the following steps of:
(a) preparing a dispersed phase by dispersing varenicline pamoate salt and a biocompatible polymer in one or more solvents;
(b) adding the prepared dispersed phase to the continuous phase and stirring to form microspheres; and
(c) removing the solvent.

All of the content described above regarding microspheres can be applied to the method for preparing microspheres of the present invention. Therefore, duplicate content will be omitted below.

The step (a) is a step of preparing a dispersed phase containing varenicline pamoate salt and a biocompatible polymer. In step (a), the varenicline pamoate salt may be dispersed or dissolved in an amount of 10 to 400 parts by weight, preferably 10 to 250 parts by weight, more preferably 10 to 100 parts by weight, and even more preferably 15 to 100 parts by weight, based on 100 parts by weight of the biocompatible polymer.

The type of solvent used to prepare the organic phase is not particularly limited, but dichloromethane, chloroform, etc. may be used.

The step (b) is a step of solidifying the microspheres by dispersing the dispersed phase prepared in step (a) into the external continuous phase to prepare an emulsion solution (O/W).

In the step (b), a hydrophilic polymer may be included as a surfactant, the type of which is not particularly limited, and any dispersed phase containing varenicline pamoate salt and biocompatible polymer may be used as long as it can help form a stable dispersed within the external continuous phase.

The hydrophilic polymer may preferably be selected from the group consisting of methylcellulose, polyvinylpyrrolidone, carboxymethylcellulose, lecithin, gelatin, polyvinyl alcohol, polyoxyethylene-polyoxypropylene block copolymer, polyoxyethylene sorbitan fatty acid ester and polyoxyethylene castor oil derivatives and mixtures thereof, and most preferably polyvinyl alcohol.

In the step (b), the external continuous phase may be an aqueous solution of 0.1 to 5% (w/v), and preferably 0.1 to 3% (w/v) of a hydrophilic polymer, where the weight average molecular weight of the hydrophilic polymer may be 10,000 to 30,000, and the degree of hydrolysis may be 80 to 90%.

In the step (b), the dispersed phase containing the varenicline pamoate salt and biocompatible polymer prepared in the step (a) is added to the external continuous phase containing the hydrophilic polymer using a drop-by-drop method or an in-line mixer, and vigorously stirred to prepare an emulsion solution (O/W). In this process, the varenicline pamoate salt is encapsulated into biocompatible polymer microspheres.

Afterwards, the solvent is removed in the step (c), and the desired microspheres can be obtained after routine filtration and washing. That is, if necessary, a step of washing the obtained microspheres using an organic solvent such as ethanol may be included to improve the Initial burst suppression effect.

In one embodiment of the present invention, the weight of varenicline pamoate salt encapsulated in the microspheres obtained according to the above production method may be 50% by weight or more, preferably 60% by weight or more, more preferably 70% by weight or more, more preferably 80% by weight or more, and most preferably 90% by weight or more, relative to the weight of the varenicline pamoate salt dissolved in step (a).

The present invention also provides a pharmaceutical composition for preventing or treating diseases caused by cholinergic receptor activity disorders, comprising the microspheres and a pharmaceutically acceptable carrier.

The diseases caused by cholinergic receptor activity disorders include, for example, inflammatory bowel disease, irritable bowel syndrome, spastic dystonia, chronic pain, acute pain, non-tropical sprue, appendicitis, vasoconstriction, anxiety disorder, panic disorder, depression, bipolar disorder, autism, sleep disorders, jet lag syndrome, amyotrophic lateral sclerosis (ALS), cognitive dysfunction, drug/toxicity-induced cognitive impairment, disease-induced cognitive impairment, hypertension, bulimia nervosa, anorexia, obesity, cardiac arrhythmias, gastric acid hypersecretion, ulcers, pheochromocytoma, progressive supranuclear palsy, chemical dependence and addiction (For example, dependence or addiction to nicotine or tobacco products, alcohol, benzodiazepines, barbiturates, opioids, or cocaine), headaches, migraines, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including absence seizures, attention deficit hyperactivity disorder (ADHD), or Tourette syndrome.

The pharmaceutical composition of the present invention can be preferably used for the prevention and treatment of nicotine dependence and addiction (smoking cessation adjuvant therapy), especially among the above diseases.

The pharmaceutical composition according to the present invention can be formulated for oral administration and can be formulated in various forms such as tablets, films, suspensions, granules, gels, pills, tinctures, decoctions, infusions, spirits, fluid extracts, elixirs, extracts, syrups, powders, aromatic waters, and lemonades. Additionally, the tablet may be formulated in various forms, such as an orally disintegrating tablet, mucoadhesive tablet, dispersible tablet, sublingual tablet, buccal tablet, chewable tablet, effervescent, and solution tablet, but is not limited to these.

The pharmaceutical composition for oral administration according to the present invention may further include a pharmaceutically acceptable carrier that can be typically added to the pharmaceutical composition.

The pharmaceutically acceptable carrier may include additives commonly used in the pharmaceutical field, such as excipients, plasticizers, disintegrants, diluents, solvents, penetration enhancers, preservatives, buffers, gel formers, lubricants, carriers, stabilizers, gels, dyes, pigments, surfactants, inert fillers, adhesives, texturizers, softeners, emulsifiers, and mixtures thereof.

The excipients include, for example, cellulose, methylcellulose, ethylcellulose, hydroxypropyl cellulose and hydroxymethylcellulose, polypropylpyrrolidone, polyvinylpyrrolidone, gelatin, gum arabic, polyethylene glycol, starch, natural and synthetic gums (e.g. gum arabic, alginate and gum arabic) and mannitol, microcrystalline cellulose, anhydrous calcium hydrogen phosphate, sorbitol, L-HPC (low-substituted hydroxypropylcellulose), pregelatinized starch, lactose and or mixtures thereof, but are not limited thereto.

The lubricants include, for example, calcium stearate, glyceryl monostearate, glyceryl palmitostearate, magnesium stearate, polyethylene glycol, sodium benzoate, sodium lauryl sulfate, sodium stearyl fumarate, stearic acid, talc, and zinc stearate, but are not limited thereto.

The disintegrants include, for example, sodium starch glycolate, sodium carboxymethylcellulose, calcium carboxymethylcellulose, croscarmellose sodium, polyvinyl pyrrolidone, methyl cellulose, microcrystalline cellulose, powdered cellulose, lower alkyl-substituted hydroxypropyl cellulose, polacrilin potassium, starch, pregelatinized starch and sodium alginate, but are not limited thereto.

In addition, the preparation for oral administration according to the present invention may, if necessary, additionally contain sweeteners, flavors, and/or colorants.

The pharmaceutical composition according to the present invention can be formulated for parenteral administration, for example, in the form of injections, creams, lotions, external ointments, oils, moisturizers, gels, aerosols, patches, and nasal inhalants, but is not limited thereto. These formulations are described in Remington's Pharmaceutical Science, 19th ed., Mack Publishing Company, Easton, PA, 1995, a commonly known text in all pharmaceutical chemistry.

The pharmaceutical composition for parenteral administration according to the present invention may contain the microspheres alone or may further include a pharmaceutically acceptable carrier for parenteral administration that can be typically added to the pharmaceutical composition. Additionally, it may additionally contain excipients or diluents. The carrier includes all types of solvents, dispersion media, oil-in-water or water-in-oil emulsions, aqueous compositions, liposomes, microbeads and microsomes.

The carrier for parenteral administration may include water, suitable oil, saline solution, aqueous glucose, glycol, etc., and may further include stabilizers and preservatives.

Suitable stabilizers include antioxidants such as sodium bisulfite, sodium sulfite or ascorbic acid. Suitable preservatives include benzalkonium chloride, methyl- or propyl-paraben and chlorobutanol.

The pharmaceutical composition of the present invention may further include lubricants, wetting agents, sweeteners, flavoring agents, emulsifiers, suspending agents, etc. in addition to the above components. Other pharmaceutically acceptable carriers and preparations can be used by referring to those described in the following literature (Remington's Pharmaceutical Sciences, 19th ed., Mack Publishing Company, Easton, PA, 1995).

The parenteral administration method of the present invention can be administered to patients (e.g., humans in need of such drugs) or other animals by intravenous, intramuscular, intraarterial, intramedullary, intrathecal, intracardiac, transdermal, subcutaneous, intraperitoneal, intranasal, enteral, intravaginal, intrapulmonary, suppository, topical, sublingual, or intrarectal administration, but is not limited to these.

In the present invention, ‘treatment’ refers to any action that improves or beneficially changes (acute or chronic) diseases, disorders, and symptoms resulting from them by administering pharmaceutical compositions. In addition, the term ‘treatment’ broadly includes the meaning of ‘prevention’, and ‘prevention’ refers to all actions that suppress or delay the onset of a disease and its symptoms by administering a pharmaceutical agent. The term ‘treatment’ includes, for example, interfering with, alleviating, improving, stopping, suppressing, delaying, reversing, etc. the progression of (acute or chronic) disease, disorder, and symptoms resulting therefrom.

The preferred total dosage of the pharmaceutical composition or drug of the present invention may be about 0.01 mg to 2,000 mg per day, most preferably 0.1 mg to 1,000 mg per day. However, the dosage, frequency and duration of administration of the pharmaceutical composition will vary depending on factors such as the nature and severity of the condition to be treated, the age and general health of the subject (host) and the subject's (host) tolerance to the active ingredients. Considering this, anyone skilled in the art will be able to determine an appropriate effective dosage of the composition of the present invention. The pharmaceutical to the composition according present invention is not particularly limited in its formulation, administration route, and administration method as long as it exhibits the effects of the present invention.

Hereinafter, the present invention will be described in detail based on the following examples. However, the following examples are only for illustrating the present invention and the scope of the present invention is not limited thereto.

### Example 1: Preparation of Varenicline Pamoate Salt

A varenicline solution was prepared by dissolving 1.00 g of varenicline (Varenicline base, manufacturer: Lee Pharma) in 50 ml of ultrapure water and adding 325 μl of acetic acid thereto. A disodium pamoate solution was prepared by completely dissolving 1.938 g of disodium pamoate (Manufacturer: Addtek chemical shanghai co LTD) in 100 ml of ultrapure water. The varenicline solution was placed in a 3-neck flask and the disodium pamoate solution was slowly added thereto while stirring at 300 rpm to obtain varenicline pamoate salt, which was freeze-dried.

### Example 2: Preparation of Varenicline Pamoate Salt-Containing Microspheres

0.25 g of varenicline pamoate salt prepared in Example 1 was dissolved in 1.1 g of dimethyl sulfoxide (manufacturer: Sigma Aldrich), and 1.0 g of biocompatible polymer (PLGA) was dissolved in 5.32 g of methylene chloride (manufacturer: Deoksan), and then mixed to complete the dispersed phase.

A 1% (w/v) polyvinyl alcohol (Sigma Aldrich, molecular weight: 13,000-23,000) aqueous solution was used as the continuous phase. 1,000 ml of the continuous phase was placed in a production tank and maintained at room temperature, and the prepared dispersed phase was injected and stirred with a homogenizer to prepare microspheres. Afterwards, the organic solvent was removed at room temperature. After washing the prepared microspheres several times with water for injection, residual polyvinyl alcohol was removed and the microspheres were freeze-dried.

### Comparative Example 1: Preparation of Varenicline Tartrate Salt-Containing Microspheres

0.1 g of varenicline tartrate (manufacturer: Viwit) and 0.9 g of biocompatible polymer (PLGA) were dissolved in 3.99 g of methylene chloride (manufacturer: Deoksan) and 3.3 g of dimethyl sulfoxide, and then mixed to complete the dispersed phase.

A 0.5% (w/v) polyvinyl alcohol (Sigma Aldrich, molecular weight: 13,000-23,000) aqueous solution was used as the continuous phase. 1,000 ml of the continuous phase was placed in a production tank and maintained at room temperature, and the prepared dispersed phase was injected and stirred with a homogenizer to prepare microspheres. Afterwards, the organic solvent was removed at room temperature. After washing the prepared microspheres several times with water for injection, residual polyvinyl alcohol was removed and the microspheres were freeze-dried.

### Comparative Example 2: Preparation of Varenicline-Containing Microspheres

0.1 g of varenicline (Varenicline base, manufacturer: Lee pharma) and 0.9 g of biocompatible polymer (PLGA) were dissolved in 4.79 g of methylene chloride (manufacturer: Deoksan) to complete the dispersed phase.

A 0.5% (w/v) polyvinyl alcohol (Sigma Aldrich, molecular weight: 13,000-23,000) aqueous solution was used as the continuous phase. 1,000 ml of the continuous phase was placed in a production tank and maintained at room temperature, and the prepared dispersed phase was injected and stirred with a homogenizer to prepare microspheres. Afterwards, the organic solvent was removed at room temperature. After washing the prepared microspheres several times with water for injection, residual polyvinyl alcohol was removed and the microspheres were freeze-dried.

### Test Example 1. Measurement of Microspheres Shape

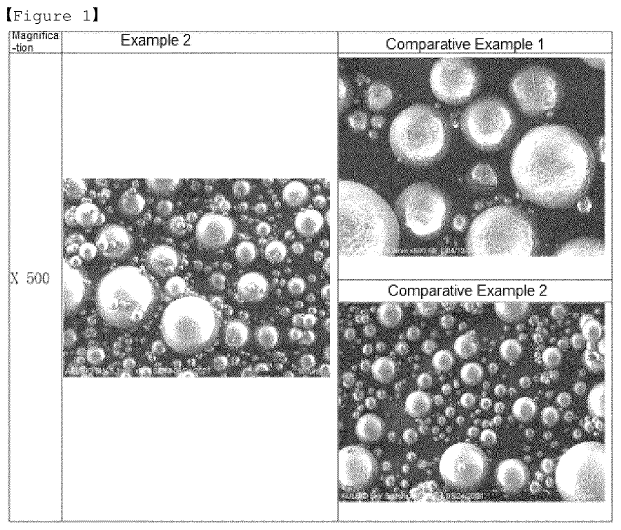

The shapes of the microspheres prepared in Comparative Examples 1 and 2, and Example 2 were analyzed using scanning electron microscopy (SEM). Approximately 20 mg of microspheres were fixed to an aluminum stub and then mounted on an SEM (equipment name: Hitachi TM4000 Plus) to observe the surface of the microspheres. All images were observed with a 5 kV electron beam at a magnification of approximately 500×. The above observation results are shown in FIG. 1.

### Test Example 2. Measurement of Varenicline Loading Amount and Encapsulation Efficiency in Microspheres

About 10 mg of the microspheres prepared in Example 2 and Comparative Examples 1 and 2 were taken, placed in a 20 mL flask, and completely dissolved in 2 mL of acetonitrile (manufacturer: Honeywell). It was adjusted to the mark with 80% methanol (manufacturer: Honeywell) and filtered through a 0.45 μm syringe filter. This solution was detected with an ultraviolet-visible spectrophotometer using HPLC (equipment name: Agilent). L1 (inner diameter 4.6 mm×150 mm, thickness 5 μm) was used as the column. The confirmation results are shown in Table 1 below.

TABLE 1

| | | Loading amount | Loading amount |
|---|---|---|---|
| | | of varenicline | of varenicline |
| | Encapsulation | pamoate salt | free base |
| | efficiency (%) | (wt %) | (wt %) |
| Comparative | 22.0 | — | 1.3 |
| Example 1 | | | |
| Comparative | 10.0 | — | 1.0 |
| Example 2 | | | |
| Example 2 | 90.9 | 18.2 | 6.4 |

From the results in Table 1, it was confirmed that the varenicline loading amount and encapsulation efficiency of the microspheres of Example 2 of the present invention were significantly superior compared to the microspheres of Comparative Example 1 and Comparative Example 2.

### Test Example 3: In Vitro Release Test and Initial Burst Evaluation

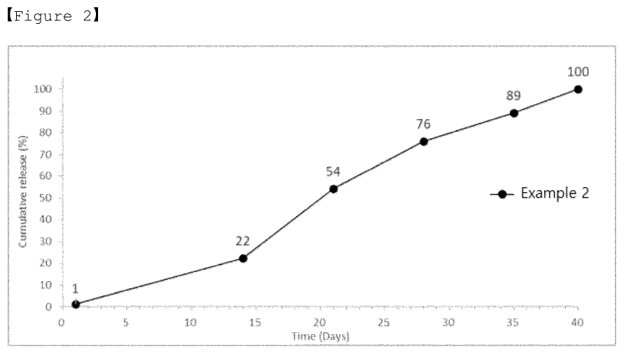

About 20 mg of the microspheres prepared in Example 2 were taken and placed in an 8 mL amber vial, and 8 mL of 0.1% poloxamer in pH 7.4 PBS solution was added thereto, stirred at 100 rpm, and maintained at 37° C. To measure the amount released over a certain period of time, 2 mL of supernatant was taken after centrifugation and filtered through a 0.22 μm RC filter. This solution was placed in a vial and detected with an ultraviolet-visible spectrophotometer using HPLC (equipment name: Agilent 1260). L1 (inner diameter 4.6 mm×150 mm, thickness 5 μm) was used as the column. The experimental results are shown graphically in FIG. 2.

From FIG. 2, it was confirmed that 51% or less of the microspheres prepared in Example 2 were released by the 20th day, and that the release continued for 40 days.

That is, from the results in FIG. 2, it was confirmed that a formulation with excellent sustained release ability could be prepared by using the varenicline pamoate salt of Example 2.

The invention claimed is:

1. Microspheres comprising varenicline or a pharmaceutically acceptable salt thereof, and a biocompatible polymer.

2. The microspheres according to claim 1, wherein the varenicline or a pharmaceutically acceptable salt thereof is contained in an amount of 0.1 to 40% by weight relative to the total weight of the microspheres based on the varenicline free base.

3. The microspheres according to claim 1, wherein the varenicline or a pharmaceutically acceptable salt thereof is varenicline pamoate salt.

4. The microspheres according to claim 3, wherein the varenicline pamoate salt is contained in an amount of 0.1 to 60% by weight based on the total weight of the microspheres.

5. The microspheres according to claim 4, wherein the biocompatible polymer is contained in an amount of 40 to 99.9% by weight based on the total weight of the microspheres.

6. The microspheres according to claim 1, wherein the biocompatible polymer is at least one selected from polylacetic acid, polylactide, polylacetic-co-glycolic acid, polylactide-co-glycolide (PLGA), polyphosphazine, polyiminocarbonate, polyphosphoester, polyanhydride, polyorthoester, copolymers of lactic acid and caprolactone, polycaprolactone, polyhydroxyvalate, polyhydroxybutyrate, polyamino acids, and copolymers of lactic acid and amino acids.

7. The microspheres according to claim 1, wherein the microspheres continue to release varenicline for more than 30 days.

8. The microspheres according to claim 1, wherein the varenicline contained in the microspheres is released at 60% by weight or less within 15 days.

9. The microspheres according to claim 1, wherein the microspheres are prepared by O/W (oil-in-water) type solvent evaporation or solvent extraction method containing a biocompatible polymer, varenicline pamoate salt, and a dispersion solvent.

10. A method for preparing microspheres comprising the following steps of:
(a) preparing a dispersed phase by dispersing varenicline pamoate salt and a biocompatible polymer in one or more solvents;
(b) adding the prepared dispersed phase to the continuous phase and homogenization to form microspheres; and
(c) removing the solvent.

11. The method for preparing microspheres according to claim 10, wherein the weight of varenicline pamoate salt encapsulated in the microspheres obtained according to the manufacturing method is 50% by weight or more compared to the weight of varenicline pamoate salt dissolved in the step (a).

12. The method for preparing microspheres according to claim 10, wherein the biocompatible polymer is at least one selected from polylacetic acid, polylactide, polylacetic-co-glycolic acid, polylactide-co-glycolide (PLGA), polyphosphazine, polyiminocarbonate, polyphosphoester, polyanhydride, polyorthoester, copolymers of lactic acid and caprolactone, polycaprolactone, polyhydroxyvalate, polyhydroxybutyrate, polyamino acids, and copolymers of lactic acid and amino acids.

13. A pharmaceutical composition comprising the microspheres of claim 1 and a pharmaceutically acceptable carrier.

14. The pharmaceutical composition according to claim 13, wherein the diseases caused by cholinergic receptor activity disorders include inflammatory bowel disease, irritable bowel syndrome, spastic dystonia, chronic pain, acute pain, non-tropical sprue, appendicitis, vasoconstriction, anxiety disorder, panic disorder, depression, bipolar disorder, autism, sleep disorders, jet lag syndrome, amyotrophic lateral sclerosis (ALS), cognitive dysfunction, drug/toxicity-induced cognitive impairment, disease-induced cognitive impairment, hypertension, bulimia nervosa, anorexia, obesity, cardiac arrhythmias, gastric acid hypersecretion, ulcers, pheochromocytoma, progressive supranuclear palsy, chemical dependence and addiction, headaches, migraines, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, attention deficit hyperactivity disorder (ADHD), or Tourette syndrome.

15. The pharmaceutical composition according to claim 13, wherein the diseases caused by cholinergic receptor activity disorders are nicotine dependence and addiction.

16. A method for preventing and/or treating a disease caused by cholinergic receptor activity disorders in a subject in need thereof, comprising administering an effective amount of the pharmaceutical composition of claim 13 to the subject.

17. The method according to claim 16, wherein the disease caused by cholinergic receptor activity disorders includes inflammatory bowel disease, irritable bowel syndrome, spastic dystonia, chronic pain, acute pain, non-tropical sprue, appendicitis, vasoconstriction, anxiety disorder, panic disorder, depression, bipolar disorder, autism, sleep disorders, jet lag syndrome, amyotrophic lateral sclerosis (ALS), cognitive dysfunction, drug/toxicity-induced cognitive impairment, disease-induced cognitive impairment, hypertension, bulimia nervosa, anorexia, obesity, cardiac arrhythmias, gastric acid hypersecretion, ulcers, pheochromocytoma, progressive supranuclear palsy, chemical dependence and addiction, headaches, migraines, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, attention deficit hyperactivity disorder (ADHD), or Tourette syndrome.

18. The method according to claim 16, wherein the disease caused by cholinergic receptor activity disorders is nicotine dependence, nicotine addiction, or a combination thereof.
